(19) United States
(12) Patent Application Publication
(10) Pub. No.: US 2007/0098797 A1
(43) Pub. Date: May 3, 2007

(54) Modified release composition of at least one form of venlafaxine

(75) Inventors: Fang Zhou, Centreville, VA (US); Werner Oberegger, Mississauga (CA); Paul Maes, Caluire et Cuire (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Biovail Laboratories International S.R.L., Mississauga (CA)

(21) Appl. No.: 11/445,198

(22) Filed: Jun. 2, 2006

Related U.S. Application Data
(60) Provisional application No. 60/686,461, filed on Jun. 2, 2005.
(60) Provisional application No. 60/691,282, filed on Jun. 17, 2005.

(51) Int. Cl. A61K 9/24

(52) U.S. Cl. 424/472

(57) ABSTRACT

The present invention relates to a modified release composition of at least one form of venlafaxine, which is a delayed controlled release composition. The composition comprises a core comprising at least one form of venlafaxine selected from the group consisting of venlafaxine, an active metabolite of venlafaxine, a pharmaceutically acceptable salt of venlafaxine, a pharmaceutically acceptable salt of an active metabolite of venlafaxine, and combinations thereof, less than 10% of a gelling agent and a pharmaceutically acceptable excipient. The composition further comprises a modified release coating which substantially surrounds the core which provides a delayed controlled release of the at least one form of venlafaxine.

### FIELD OF THE INVENTION

The present invention relates to modified release compositions for oral administration of at least one form of venlafaxine, to processes for their preparation and to their medical use. In particular, the modified release composition relates to a delayed controlled release composition of at least one form of venlafaxine.

### BACKGROUND OF THE INVENTION

An ideal dosage regimen for many medications is that by which an acceptable therapeutic concentration of drug at the site(s) of action is attained immediately and is then maintained constant for the duration of the treatment. Providing dose size and frequency of administration are correct, therapeutic “steady-state” plasma concentrations of a drug can be achieved promptly and maintained by the repetitive administration of conventional peroral dosage forms. However, there are a number of potential limitations associated with conventional peroral dosage forms. These limitations have led pharmaceutical scientists to consider presenting therapeutically active molecules in “extended-release” preparations.

Oral ingestion is the traditionally preferred route of drug administration, providing a convenient method of effectively achieving both local and systemic effects. An ideal oral drug delivery system should steadily deliver a measurable and reproducible amount of drug to the target site over a prolonged period. Extended-release (ER) delivery systems provide a uniform concentration/amount of the drug at the absorption site and thus, after absorption, allow maintenance of plasma concentrations within a therapeutic range over an extended period of time, which can minimize side effects and also reduces the frequency of administration. ER dosage forms release drug slowly, so that plasma concentrations are maintained at a therapeutic level for a prolonged period of time. Typically, these products provide numerous benefits compared with immediate-release compositions, including greater effectiveness in the treatment of chronic conditions, reduced side effects, greater convenience, and higher levels of patient compliance due to a simplified dosing schedule. Because of the above advantages, such systems form a major segment of the drug delivery market.

Many drug delivery systems have been developed with the aim of eliminating the cyclical changes in plasma drug concentration seen after the administration of a conventional delivery system. A variety of terms have been used to describe these systems: delayed release, repeat action, prolonged release, sustained release, extended release, controlled release and modified release. It is interesting to note that the USP considers that the terms controlled release, prolonged release, sustained release and extended-release are interchangeable.

Controlled-release formulations have been described in the prior art and many methods have been used to provide controlled-release pharmaceutical dosage forms in order to maintain therapeutic serum levels of medicaments and to minimize the effects of missed doses of drugs caused by a lack of patient compliance. Anti-depressants are excellent candidates for controlled-release formulations as discontinuation of these drugs, most often as a result of a lack of patient compliance due to a complicated or multiple daily dosing schedule, can often result in severe discontinuation symptoms.

Venlafaxine, chemically designated as (R/S)-1-[2-(dimethylamino)-1-(4-methoxyphenyl)ethyl] cyclohexanol or (±)-1-[a [α-(dimethylaminio)methyl] p-methoxybenzyl] cyclohexanol, is a bicyclic compound with antidepressant properties affecting chemical messengers within the brain. These chemical messengers, called neurotransmitters, can for example be serotonin, dopamine, and norepinephrine. Neurotransmitters are manufactured and released by nerve cells. The neurotransmitters travel to neighboring nerve cells and cause the cells to become more or less active. It is believed that an imbalance in these neurotransmitters is the cause of depression and also may play a role in anxiety. Venlafaxine is believed to work by inhibiting the release or affecting the action of these neurotransmitters.

Venlafaxine is chemically unrelated to other antidepressants, but is sometimes categorized as a serotonin-norepinephrine reuptake inhibitor (SNRI). At low dosages, venlafaxine blocks serotonin reuptake, similarly to a selective serotonin reuptake inhibitor (SSRI). At medium dosages, venlafaxine blocks the reuptake of norepinephrine as well as serotonin. At high dosages, venlafaxine blocks the reuptake of norepinephrine, serotonin and is also a weak blocker of the reuptake of dopamine.

Venlafaxine is well absorbed after oral administration and its metabolism has been well documented. Following absorption, venlafaxine undergoes extensive pre-systemic metabolism in the liver, primarily to O-desmethylvenlafaxine (ODV), but also to N-desmethylvenlafaxine (NDV), N,O-didesmethylvenlafaxine (DDV), and N,N,O-tridesmethylvenlafaxine (TDV). In vitro studies indicate that the formation of ODV is catalyzed by CYP2D6; this has been confirmed in a clinical study showing that patients with low CYP2D6 levels (“poor metabolizers”) had increased levels of venlafaxine and reduced levels of ODV compared to people with normal levels of CYP2D6 (“extensive metabolizers”). The differences between CYP2D6 poor and extensive metabolizers, however, are not expected to be clinically important because the sum of venlafaxine and ODV is similar in the two groups and venlafaxine and ODV are pharmacologically approximately equiactive and equipotent. Approximately 87% of a venlafaxine dose is recovered in the urine within 48 hours as unchanged venlafaxine (5%), unconjugated ODV (29%), conjugated ODV (26%), or other minor active metabolites (27%). Renal elimination of venlafaxine and its metabolites is the primary route of excretion. The metabolic pathway of venlafaxine can be summarized as follows:

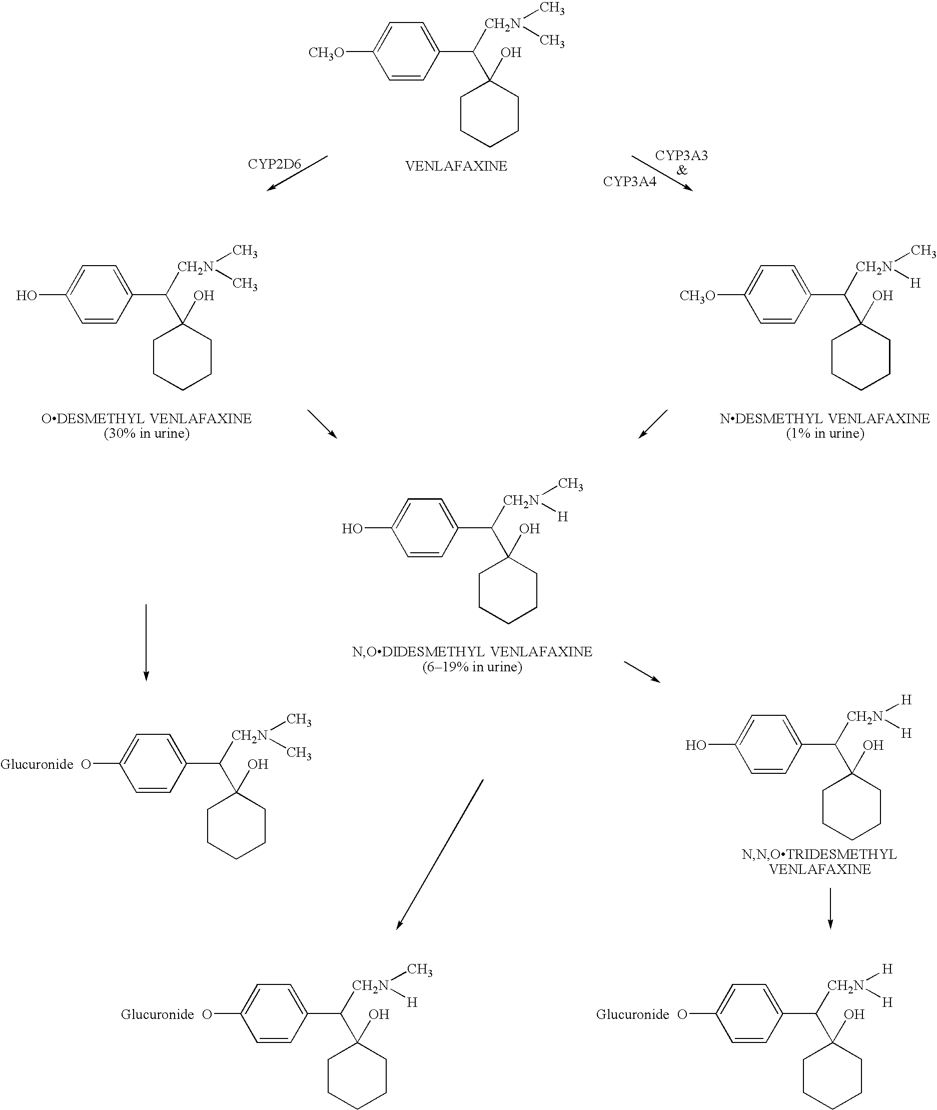

Venlafaxine's elimination half-life of about 4 hours is short, and its active metabolite has a half-life of about 8 hours. This results in venlafaxine being administered twice daily and a lack of patient compliance in keeping to this daily dosing schedule is liable to produce discontinuation problems. Sudden discontinuation of venlafaxine can result in withdrawal symptoms, which can include, fatigue, dizziness, nausea, headache and dysphoria. Accordingly, venlafaxine is an excellent candidate for a controlled-release oral formulation.

Venlafaxine, as its hydrochloride salt, is available as a second-generation extended-release tablet and is marketed under the brand name Effexor® XR for once daily use. Such a formulation has eliminated the discontinuation problems seen with Effexor®, the first-generation immediate-release form of venlafaxine, which is usually administered twice daily. Extended-release formulations of venlafaxine have been described in the prior art.

U.S. Pat. Nos. 6,274,171, 6,403,120, and 6,419,958, for example, disclose formulations comprising a therapeutically effective amount of venlafaxine hydrochloride in film-coated spheroids. The spheroids comprise a core having venlafaxine hydrochloride, microcrystalline cellulose, and optionally hydroxypropylmethylcellulose. The cores are coated with a mixture of ethylcellulose and hydroxypropylmethylcellulose and subsequently packaged into hard gelatin capsules. These patents also describe and claim methods and compositions for obtaining therapeutic blood plasma concentrations of venlafaxine over a twenty-four hour period with diminished incidence of nausea and emesis which comprise administering orally to a patient in need thereof, an extended-release formulation providing a peak blood plasma level of venlafaxine of no more than about 150 ng/ml 4-8 hours after administration.

U.S. Pat. No. 6,703,044 purports to teach a formulation wherein a delayed-burst release of venlafaxine is achieved at least three hours after administration resulting in dispersion of the venlafaxine mainly through the colon into the blood stream as a result of colon absorption over a period of at least 24 hours. A compressed core comprising a burst controlling agent as well as a disintegrant characterizes the formulation. The core is coated with a relatively rigid water insoluble, hydrophobic polymer, in which particles of water insoluble but hydrophilic material are embedded. These particles form channels upon contact with aqueous medium, which imbibe liquid and cause the burst-controlling agent to burst the coating thereby enabling the delayed-burst release of the venlafaxine. The '044 patent also teaches in Example 11 that the formulation surprisingly provided for a 30% higher bioavailability of the venlafaxine in fasting volunteers when compared to extended-release formulations of venlafaxine presently available on the market. The label for Effexor® XR, on the other hand, states that: “Effexor XR should be administered in a single dose with food either in the morning or evening at approximately the same time each day”. Example 11, the only pharmacokinetic study presented in the patent, does not show any bioavailability data in fed volunteers, and hence it is not known whether the formulation taught in the '044 patent will also provide for a higher bioavailability when administered to patients under the conditions recommended by the Effexor® XR label, i.e. under fed conditions. The '044 patent does not provide any data on the adverse events or side effect profile of the claimed composition.

The disclosures of the '120, '171, and '958 patents discussed above teach that “ . . . various attempts to produce extended release tablets of venlafaxine hydrochloride by hydrogel technology proved to be fruitless because the compressed tablets were either physically unstable (poor compressibility or capping problems) or dissolved too rapidly in dissolution studies.” Col. 4, lines 60-64 of the '120, 171, and '958 patents. Makhija and Vavia of the Pharmaceutical Division, Dept. of Chemical Technology (Autonomous), University of Mumbai, India, however, describe a once daily sustained-release tablet of venlafaxine using hydrogel technology (Eur. J. Pharmaceut. Biopharmaceut. 2002. 54:9-15). The Makhija and Vavia reference teaches a once daily sustained-release tablet of venlafaxine hydrochloride using an uncoated matrix system based on swellable as well as non-swellable polymers. Interestingly, the bioavailability of venlafaxine for this formulation, like that of the '044 formulation is, also significantly improved over that of Effexor® XR even though there does not appear to be any delay in the release of the drug in vitro (FIG. 2) or in vivo (FIG. 4). However, like the '044 invention, the formulation was administered to individuals in the fasted state. Accordingly, it is not known whether the Makhija and Vavia formulation would provide a higher bioavailability in the fed state. Finally, the Makhija and Vavia reference does not teach the effect of their formulation on the incidence and frequency of any adverse events in comparison to Effexor® XR.

Delayed release formulations comprising venlafaxine as the active agent have also been described in the prior art. For example, U.S. patent application Ser. No. 10/244,059, published as US 2003/0091634A1 on May 15, 2003 and U.S. patent application Ser. No. 09/953,101, published as US 2003/0059466A1 on Mar. 27, 2003 both describe a delayed release tablet, comprising a core comprising 10 to 70% of active agent, 10 to 80% of a gelling agent, and optional conventional excipients; and a coating consisting essentially by weight, based on the coating weight, of 20 to 85% of a water-insoluble, water-permeable film-forming polymer, of 10 to 75% of a water-soluble polymer or substance and 3 to 40% of a plasticizer.

Venlafaxine is currently among the top five prescribed antidepressant medications within the SSRI/SNRI category of antidepressants. However, only one once-a-day oral dosage form comprising venlafaxine hydrochloride is currently being marketed under the trade name Effexor® XR. Given the efficacy of venlafaxine, a once-a-day oral composition comprising at least one form of venlafaxine capable of providing a higher bioavailability compared to the currently marketed, Effexor®150 mg capsules, with a reduced or similar side effect or adverse event profile would be desirable. Such a composition can also allow for a composition having an absolute amount of the active drug that is less that the amount in the reference product, thereby providing for a better safety profile.

### SUMMARY OF THE INVENTION

The present invention relates to a modified release composition of at least one form of venlafaxine.

In one embodiment of the invention, the modified release composition of the at least one form of venlafaxine is a delayed controlled release pharmaceutical composition for oral administration suitable for once daily dosing comprising: a) a core comprising by weight of the core dry weight from about 10% to about 90% of at least one form of venlafaxine selected from the group consisting of venlafaxine, a pharmaceutically acceptable salt of venlafaxine, an active metabolite of venlafaxine, a pharmaceutically acceptable salt of an active metabolite of venlafaxine, and combinations thereof, less than 10% of a gelling agent, and optional conventional excipients, and b) a modified release coat substantially surrounding said core; wherein said composition provides a delayed controlled release of said at least one form of venlafaxine such that no more that 20% of the at least one form of venlafaxine is released after about 2 hours, about 15% to about 45% of the at least one form of venlafaxine is released after about 4 hours, about 55% to about 85% of the at least one form of venlafaxine is released after about 8 hours, no less than about 65% of the at least one form of venlafaxine is released after about 12 hours and no less than about 80% of the at least one form of venlafaxine is released after about 16 hours when tested using USP Apparatus 1 in 1000 ml of pH 6.8 phosphate buffer at 75 rpm at 37° C.±0.5° C.

In another embodiment of the invention, the modified release composition of the at least one form of venlafaxine is a delayed controlled release pharmaceutical composition for oral administration suitable for once daily dosing comprising: a) a core comprising by weight of the core dry weight from about 10% to about 90% of at least one form of venlafaxine selected from the group consisting of venlafaxine, a pharmaceutically acceptable salt of venlafaxine, an active metabolite of venlafaxine, a pharmaceutically acceptable salt of an active metabolite of venlafaxine, and combinations thereof, less than 10% of a gelling agent, and optional conventional excipients; and b) a modified release coat substantially surrounding said core, said coat comprising by weight of the coat dry weight from about 20% to about 85% of a water-insoluble water-permeable film-forming polymer, from about 10% to about 75% of a water-soluble polymer or substance and from about 3% to about 40% of a plasticizer; wherein said composition provides a delayed controlled release of said at least one form of venlafaxine such that no more that 20% of the at least one form of venlafaxine is released after about 2 hours, about 15% to about 45% of the at least one form of venlafaxine is released after about 4 hours, about 55% to about 85% of the at least one form of venlafaxine is released after about 8 hours, no less than about 65% of the at least one form of venlafaxine is released after about 12 hours and no less than about 80% of the at least one form of venlafaxine is released after about 16 hours when tested using USP Apparatus 1 in 1000 ml of pH 6.8 phosphate buffer at 75 rpm at 37° C.±0.5° C.

### DETAILED DESCRIPTION OF THE INVENTION

The present invention is directed to a modified release pharmaceutical composition of venlafaxine. In particular, the composition is an enhanced absorption delayed controlled release composition of the at least one form of venlafaxine comprising a core and a modified release coating, which substantially surrounds the core, wherein the composition provides a delayed controlled release of the at least one form of venlafaxine. The enhanced absorption delayed controlled release oral dosage form of the invention has a higher bioavailability with reduced or similar side effects or adverse events when compared to the reference product.

The Tablet Cores

The core comprises at least one form of venlafaxine selected from the group consisting of venlafaxine, a pharmaceutically acceptable salt of venlafaxine, an active metabolite of venlafaxine, a pharmaceutically acceptable salt of an active metabolite of venlafaxine, and combinations thereof, a gelling agent and optionally conventional excipients, surrounded by a modified release polymer coat. The composition provides a delayed controlled release of the at least one form of venlafaxine.

The proportion of the at least one form of venlafaxine in the core is present from about 10 to about 90%, preferably from about 20 to about 60%, more preferably from about 35% to about 45%, and most preferably about 42% by weight of the core dry weight. The composition comprises a pharmaceutically effective amount of the at least one form of venlafaxine that can vary from about 0.5 to about 1000 mg, more preferably from about 20 to about 200 mg, and most preferably from about 100 to about 200 mg.

The term “effective amount” as used herein means that a “pharmaceutically effective amount” is contemplated. A “pharmaceutically effective amount” is the amount or quantity of the at least one form of venlafaxine in a dosage form of the invention sufficient to elicit an appreciable clinical or therapeutic response when administered, in single or multiple doses to a patient in need thereof. It will be appreciated that the precise therapeutic dose will depend on the age and condition of the patient and the nature of the condition to be treated and will be at the ultimate discretion of the attendant physician. It is well known to the skilled artisan that the therapeutically or clinically effective amount for a certain indication can be determined by conducting clinical studies using dosage forms that contain a pharmaceutically effective amount of the at least one form of venlafaxine.

As used herein, the term “pharmaceutically acceptable salts” refers to salts prepared from pharmaceutically acceptable non-toxic acids, including inorganic acids and organic acids. Suitable non-toxic acids include inorganic and organic acids such as acetic, benzenesulfonic, benzoic, camphorsulfonic, citric, ethenesulfonic, fumaric, gluconic, glutarnic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesunfonic and the like. The hydrochloric salt is the most preferred. Other salts, such as venlafaxine maleate and venlafaxine besylate have been described in International patent application Nos. PCT/EP03/03319 (WO 03/082805) and PCT/EP03/03318 (WO 03/082804) respectively, the contents of which are incorporated herein by reference.

Venlafaxine, or the venlafaxine in the pharmaceutically acceptable salts of venlafaxine, can be any form of venlafaxine. For example, venlafaxine has one optically active carbon, thus allowing for existence of two enantiomers and a racemate. Both enantiomers are pharmaceutically active. Thus, the effective amount of the preferred active in the core of the oral dosage form of the invention, venlafaxine hydrochloride, can be based on the racemate or mixture of enantiomers of venlafaxine or on the pure or substantially pure (+) or (−) enantiomer of venlafaxine. The (+) and (−) enantiomers of venlafaxine have been described in U.S. Pat. Nos. 6,197,828 and 6,342,533 respectively the contents of which are incorporated herein by reference. All such forms of venlafaxine are included within the meaning of the term “venlafaxine”, “pharmaceutically acceptable salts of venlafaxine”, “active metabolite of venlafaxine”, and “pharmaceutically acceptable salts of an active metabolite of venlafaxine”.

The at least one gelling agent comprises a substance that is hydrophilic in nature and which is capable of behaving like a hydrophilic matrix. Examples of gelling agents are described in U.S. patent Ser. No. 10/244,059, published May 15, 2003 as US 2003/0091634 and in the Handbook of Pharmaceutical Excipients, 4^{th} Edition (2003), edited by Rowe et al. and published by the Pharmaceutical Press and the American Pharmaceutical Association. The at least one gelling agent is present in an amount less than 10%, preferably about less than 8%, more preferably about less than 6%, even more preferably about less than 4% and most preferably about less than 2% by weight of the core dry weight. The ideal gelling agent is polyvinyl alcohol present at about 1.5% (for a 180, 120, or 60 mg tablet of venlafaxine) or about 1% (for a 30 mg tablet of venlafaxine) by weight of the core dry weight. The at least one gelling agent can comprise a mixture of two or more gelling agents as long as the total amount of the gelling agent is less than 10% of the core dry weight. One example of a combination of gelling agents can comprise a mixture of polyvinyl alcohol at about 1.5% and methylcellulose at about 5% or polyvinyl alcohol at about 3% and methylcellulose at about 5% by weight of the core dry weight.

Without wishing to be bound to any particular theory it is believed that a low gelling formulation i.e., a formulation having less than 10% of a gelling agent, in addition to the enhanced absorption delayed controlled release characteristic of the composition described herein, can contribute or further improve on the safety profile. Venlafaxine is first and foremost a serotonin uptake or reuptake inhibitor (SRI). At higher doses/concentrations, venlafaxine also inhibits the uptake of norepinephrine. Because the doses at which both these activities occur are relatively close together, venlafaxine is also referred to as a serotonin/norepinephrine reuptake inhibitor (SNRI). However, the SRI portion is more important for discussions of nausea and emesis. The action of venlafaxine to block the uptake of serotonin will result in the elevation of serotonin in all synapses exposed to venlafaxine. Synapses occur not only between neurons in the central nervous system, but also between neurons and muscle fibers i.e., the neuromuscular junction. It is known that the stomach has serotonin receptors. Cohen and Fludzinski, JPET 1987; 243:264-269. Therefore, and without wishing to be bound to any particular theory, release of venlafaxine in the stomach can produce elevations of serotonin at synapses surrounding the stomach resulting in turn in nausea and emesis. Theoretically, reducing the gelling agent to less than 10% can result in a composition, which would not swell at all or at least not as much as a composition having a gelling agent greater than 10%. Such a low gelling composition can be expected to have a shorter retention time in the stomach, resulting in much less release of venlafaxine in the stomach as compared to a composition having greater than 10% gelling agent. The lower amounts of venlafaxine in the stomach can result in lower elevations of serotonin at synapses surrounding the stomach and in turn cause fewer gastrointestinal related side effects or adverse events such as nausea and emesis.

In addition to the above ingredients, a series of excipients can be included in the tablet to ensure that the tableting operation can run satisfactorily and to ensure that tablets of specified quality are prepared. Depending on the intended main function, excipients to be used in tablets are subcategorized into different groups. However, one excipient can affect the properties of a tablet in a series of ways, and many excipients used in tablet compositions can thus be described as being multifunctional.

For example, the core can further comprise at least one lubricant. Lubricants are added to pharmaceutical formulations to ensure that tablet formation and ejection can occur with low friction between the solid and the die wall. High friction during tabletting can cause a series of problems, including inadequate tablet quality (capping or even fragmentation of tablets during ejection, and vertical scratches on tablet edges) and can even stop production. Non-limiting examples of lubricants useful for the oral dosage form described herein include magnesium stearate, talc, sodium stearyl fumarate, calcium stearate, silica gel, colloidal silicon dioxide, Compritol 888 ATO, glyceryl behenate, stearic acid, hydrogenated vegetable oils (such as hydrogenated cottonseed oil (Sterotex®), hydrogenated soybean oil (Sterotex® HM) and hydrogenated soybean oil & castor wax (Sterotex® K), stearyl alcohol, leucine, polyethylene glycol (MW 4000 and higher), and mixtures thereof. The at least one lubricant can be present in an amount from about 0.02 to about 5% by weight of the core dry weight. The preferred lubricant is glyceryl behenate and is preferably present at about 3% by weight of the core dry weight.

Some oral dosage forms require the incorporation of one or more excipients into the dosage form to increase the bulk volume of the powder and hence the size of the dosage form. Accordingly, the core can further comprise at least one filler (or diluent). Non-limiting examples of the at least one filler useful for the oral dosage form described herein include lactose monohydrate, anhydrous lactose, mannitol, sorbitol, microcrystalline cellulose, dibasic calcium, and calcium sulfate. Mixtures of fillers can also be used. The at least one filler is preferably present up to about 75% by weight of the core dry weight. The preferred filler is lactose monohydrate. Most preferably, the lactose monohydrate is of the type called Lactose #315 Spray Dried, which is a mixture of a specially prepared pure α-lactose monohydrate along with a small amount of amorphous lactose. Preferably, the Lactose #315 Spray Dried is present at about 53% (for a 180, 120, or 60 mg tablet of venlafaxine) or 72% (for a 30 mg tablet of venlafaxine) by weight of the core dry weight.

The at least one form of venlafaxine and filler, preferably Lactose 315 (Spray Dried) are first granulated with an aqueous solution of the gelling agent, preferably polyvinyl alcohol, in a suitable fluid bed granulator apparatus. The granulate is subsequently dried and sieved through a 1.4 mm screen. The sized granules are next blended with more filler in a V-blender or any other suitable blending apparatus together with a lubricant, preferably glyceryl behenate, and if necessary, any other additional inert excipients, which can improve processing of the oral dosage form of the invention. Alternatively, the ingredients can also be dry blended and directly compressed by methods known in the art.

The dried milled granules are then pressed into tablets and are hereinafter referred to as “tablet cores” or simply as “cores”. Tablet cores can be obtained by the use of standard techniques and equipment well known to the skilled artisan. Preferably, the tablet cores are obtained by a rotary press (also referred to as a multi-station press) fitted with suitable punches. At this stage, the core formulation is an immediate-release formulation resulting in greater than about 90% release of the at least one form of venlafaxine in about 30 minutes.

The Coat

The cores are next coated with a polymer coat designed to achieve a delayed controlled-release of the at least one form of venlafaxine. The coat is designed to achieve an in vitro release profile of the at least one form of venlafaxine, preferably the hydrochloride salt of venlafaxine, such that the composition, when tested in vitro using the USP type I method at 75 rpm in 1000 ml phosphate buffer pH 6.8 at 37° C. releases no more that 20% of the at least one form of venlafaxine after about 2 hours, about 15% to about 45% of the at least one form of venlafaxine after about 4 hours, about 55% to about 85% of the at least one form of venlafaxine after about 8 hours, no less than about 65% of the at least one form of venlafaxine is after about 12 hours and no less than about 80% of the at least one form of venlafaxine after about 16 hours.

The preferred polymer coat for achieving the delayed controlled-release of the at least one form of venlafaxine is a semi-permeable coat permeable to venlafaxine and does not have a preformed pore as described for example in U.S. Pat. No. 5,654,005. The semi-permeable coat comprises at least one water-insoluble, water-permeable film-forming polymer, at least one water-soluble polymer or substance, and at least one plasticizer. The polymer coat is designed such that the integrity of the coat remains intact and does not dissolve and/or disintegrate for a period of at least about 24 hours in purified water, 0.1 N HCl, Simulated Gastric Fluid (SGF) pH 1.2, or pH 6.8 phosphate buffer. As these conditions are intended to mimic the in vivo condition, it is believed that the integrity of the polymer coat will also remain intact and not dissolve and/or disintegrate in the gastrointestinal tract. The polymer coat described herein is thus fundamentally different from the polymer coat described in U.S. Pat. No. 6,117,453, which is a quick-dissolving film, and U.S. Pat. No. 6,703,044, which is a rigid film designed to burst, thereby releasing the active from the core.

Non-limiting examples of the at least one water-insoluble, water permeable film-forming polymer can be a cellulose ether, such as ethylcellulose, a cellulose ester, such as cellulose acetate, methacrylic acid derivatives, aqueous ethylcellulose dispersions such as Surelease®, aqueous enteric coating systems such as Sureteric®, and aqueous acrylic enteric systems such as Acryl-EZE®. Combinations are also permitted. The at least one water-insoluble, water-permeable film forming polymer is present in an amount ranging from about 20 to about 85%, preferably from about 55 to about 62%, and most preferably about 60% by weight of the coating dry weight. Most preferably, ethylcellulose is the at least one water-insoluble, water-permeable film-forming polymer and is preferably present from about 55 to about 62% and most preferably at about 60% of the coating dry weight.

The at least one water-soluble polymer or substance can be a partially or totally water-soluble hydrophilic substance intended to modulate the film permeability to the outside aqueous medium. Non-limiting examples of the at least one water-soluble polymer or substance can be polyvinylpyrrolidone, polyethyleneglycol, hydroxypropylmethylcellulose, hydrated colloidal silica, sucrose, mannitol, and combinations thereof. The at least one water-soluble polymer comprises from about 10 to about 75%, preferably from about 20% to about 30% and most preferably about 23% to about 26% by weight of the coating dry weight. Most preferably, the at least one water-soluble polymer is polyvinylpyrrolidone and comprises preferably from about 23% to about 26% by weight of the coating dry weight.

Plasticizers are generally added to film coating formulations to modify the physical properties of the polymer to make it more usable. The amount and choice of the plasticizer contributes to the hardness of a tablet and may even affect its dissolution or disintegration characteristics, as well as its physical and chemical stability. One important property of plasticizers is their ability to make a coat elastic and pliable, thereby decreasing the coat's brittleness. Non-limiting examples of the at least one plasticizer useful for the preferred polymer coat include polyols, such as polyethylene glycol of various molecular weights, organic esters, such as diethyl phthalate or triethyl citrate, dibutyl sebacate, dibutyl pthalate, and oils/glycerides such as fractionated coconut oil or castor oil. Combinations are permitted. The at least one plasticizer is present from about 3 to about 40%, preferably from about 13 to about 18%, and most preferably about from about 15% to about 17% by weight of the coating dry weight. The preferred at least one plasticizer is dibutyl sebacate, and is preferably present in an amount from about 15% to about 17% by weight of the coating dry weight.

The relative proportions of the preferred polymer coat ingredients, notably the ratio of the at least one water-insoluble, water-permeable film-forming polymer:the at least one water-soluble polymer or substance:the at least one plasticizer, can be varied depending on the desired rate of release. The skilled artisan will appreciate that controlling the permeability and/or the amount of coating applied to the tablet cores can control the rate of release of the active. For example, the permeability of the preferred polymer coat, can be altered by varying the ratio of the at least one water-insoluble, water-permeable film-forming polymer:the at least one water-soluble polymer:the at least one plasticizer and/or the quantity of coating applied to the tablet cores. A more delayed controlled-release is generally obtained with a higher amount of water-insoluble, water-permeable film forming polymer, a lower amount the at least one water soluble polymer, and/or by increasing the amount of the coating solution applied to the tablet cores. Alternatively, a faster rate of release can be obtained by increasing the amount of the water-soluble polymer, decreasing the amount of the at least one water-insoluble water permeable film-forming polymer, and/or by decreasing the amount of coating solution applied. The addition of other excipients to the tablet core can also alter the permeability of the coat. For example, if it is desired that the tablet core further comprise an expanding agent, the amount of plasticizer in the coat can be increased to make the coat more pliable as the pressure exerted on a less pliable coat by the expanding agent can rupture the coat. Other excipients such as pigments and taste-masking agents can also be added to the coating formulation. The preferred proportions of the at least one water-insoluble water-permeable film forming polymer:the at least one water-soluble polymer:the at least one plasticizer for maintaining the integrity of the coat for at least about 24 hours and for obtaining the in vitro release profile described above is about 50-85:10-40:5-20. Preferably the ratio is about 58-60:23-26:15-17.

The polymer coat was prepared and applied as follows. The appropriate amounts of the water-insoluble water-permeable film-forming polymer, preferably ethylcellulose, the water-soluble polymer, preferably, polyvinylpyrrolidone, and plasticizer, preferably dibutyl sebacate were all dissolved in an alcoholic solvent such as ethanol, isopropyl alcohol, or a mixture thereof. The resulting coating solution was sprayed onto the tablet cores, using a coating pan apparatus. The percentage weight gain resulting from application of the coating solution onto the cores can range from about 2 to about 50%, preferably from about 8 to about 30%, more preferably from about 10 to about 18% and most preferably from about 12% to about 15% by weight of the uncoated cores. Surprisingly, it was discovered that the above coating formulation provides for a delayed controlled-release composition even though no monomeric pore-forming agent is present in the coating and the core has less than 10% of a gelling agent.

The following examples illustrate the present invention and are not intended to limit the scope of the present invention.

### EXAMPLE 1

30 mg Venlafaxine Delayed Controlled Release Tablets

The materials shown in Table 1 were combined to produce tablet cores for 30 mg venlafaxine delayed controlled release tablets:

TABLE 1

| Ingredients | Mg | % w/w |
|---|---|---|
| Venlafaxine Hydrochloride, USP | 33.95 | 24 |
| Gelling Agent¹ | 1.21 | 0.9 |
| Filler² | 100.64 | 72 |
| Lubricant³ | 4.2 | 3 |
| Purified Water⁴, USP | N/A | N/A |
| Tablet Core Weight | 140 | 100 |

¹Polyvinyl Alcohol, USP
²Lactose #315 Spray Dried, USP
³Glyceryl Behenate, NF
⁴Evaporates after drying

The venlafaxine hydrochloride and filler, Lactose 315 (Spray Dried), were first granulated with an aqueous solution of the gelling agent, polyvinyl alcohol, in a suitable fluid bed granulator apparatus. The granulate was subsequently dried and sieved through a 1.4 mm screen. The sized granules were next blended with more filler together with the lubricant, glyceryl behenate, in a V-blender and then compressed into tablets using a conventional rotary tablet press.

The dissolution of the resulting tablet cores was determined under the following conditions:
Medium: 1000 ml pH 6.8 phosphate buffer
Method: USP Type I Apparatus, 75 rpm at 37° C.±0.5° C.
   The data showed that greater than 90% of the venlafaxine hydrochloride is released in about 30 minutes.

The materials shown in Table 2 were combined to produce the modified release coat:

TABLE 2

| Ingredients | Mg | % w/w |
|---|---|---|
| Water-insoluble water-permeable | 12.6 | 60 |
| film forming polymer¹ | | |
| Water-soluble polymer² | 4.9 | 23.3 |
| Plasticizer³ | 3.5 | 16.6 |
| Solvent⁴ | N/A | N/A |
| Total Dry Solids (% weight gain) | 21 (15) | 100 |
| Tablet Cores | 140 | — |
| Total Weight of Coated Tablet | 161 | — |

¹Ethylcellulose 100, NF
²Povidone, USP
³Dibutyl Sebacate, NF
⁴Ethyl Alcohol (200 proof), USP and Isopropyl Alcohol (99%), USP, both evaporate after drying

The plasticizer, dibutyl sebacate, was first dissolved in the solvent (ethyl alcohol/isopropyl alcohol mixture). The water-insoluble water-permeable film-forming polymer (Ethylcellulose) was slowly added to the plasticizer/solvent mixture followed by the addition of the water-soluble polymer (Povidone) until a homogenous solution was achieved. Coating of the tablet cores from Example 1 was then carried out in an O'Hara Labcoat III System until an about 15% weight gain was achieved.

The tablets were coated until the desired weight gain was reached and subsequently dried at an inlet air temperature set at 50±3° C., for 5 minutes at pan speed 2 rpm. Drying was continued for another 40 minutes at Jog with the same pan speed and the same parameters. The inlet temperature was subsequently turned off and the tablets cooled by keeping the exhaust on. The dissolution of the coated tablets was determined under the same experimental conditions as for the uncoated tablet cores. The results are presented in Table 3 as % released of the total venlafaxine hydrochloride in the coated tablet cores:

TABLE 3

| Time (hr) | % Released |
|---|---|
| 1 | 4 |
| 2 | 15 |
| 3 | 26 |
| 4 | 37 |
| 5 | 48 |
| 6 | 57 |
| 7 | 64 |
| 8 | 71 |
| 9 | 76 |
| 10 | 80 |
| 11 | 84 |
| 12 | 86 |
| 13 | 89 |
| 14 | 91 |
| 15 | 93 |
| 16 | 95 |
| 17 | 97 |
| 18 | 98 |
| 19 | 99 |
| 20 | 100 |
| 21 | 100 |
| 22 | 101 |
| 23 | 101 |
| 24 | 102 |

The release profile of the coated tablet cores compared to the release profile of the uncoated cores shows that the polymers if used in the granulation process to form the cores do not significantly impede the release of drug from the tablet. The polymer coat provides the delayed controlled release profile. This is also true for all dosage strengths of venlafaxine.

### EXAMPLE 2

60 mg Venlafaxine Delayed Controlled Release Tablets

The materials shown in Table 4 were combined to produce tablet cores for 60 mg venlafaxine delayed controlled release tablets:

TABLE 4

| Ingredients | Mg | % w/w |
|---|---|---|
| Venlafaxine Hydrochloride, USP | 67.90 | 42 |
| Gelling Agent¹ | 2.4 | 1.5 |
| Filler² | 84.90 | 53 |
| Lubricant³ | 4.8 | 3 |
| Purified Water⁴, USP | N/A | N/A |
| Tablet Core Weight | 160 | 100 |

¹Polyvinyl Alcohol, USP
²Lactose #315 Spray Dried, USP
³Glyceryl Behenate, NF
⁴Evaporates after drying

The tablet cores were manufactured as described in Example 1 and Subsequently coated as also described in Example 1 with a solution of materials shown in Table 5:

TABLE 5

| Ingredients | Mg | % w/w |
|---|---|---|
| Water-insoluble water-permeable | 11.4 | 60 |
| film forming polymer¹ | | |
| Water-soluble polymer² | 4.43 | 23.3 |
| Plasticizer³ | 3.17 | 16.6 |
| Solvent⁴ | N/A | N/A |
| Total Dry Solids (% weight gain) | 19(12) | 100 |
| Tablet Cores | 160 | — |
| Total Weight of Coated Tablet | 179 | — |

¹Ethylcellulose 100, NF
²Povidone, USP
³Dibutyl Sebacate, NF
⁴Ethyl Alcohol (200 proof), USP and Isopropyl Alcohol (99%), USP, both evaporate after drying

The dissolution of the coated tablets was determined as described in Example 1 for the uncoated tablet cores. The results are presented in Table 6 as % released of the total venlafaxine hydrochloride coated tablet cores:

TABLE 6

| Time | % Released |
|---|---|
| 1 | 3 |
| 2 | 11 |
| 3 | 21 |
| 4 | 30 |
| 5 | 40 |
| 6 | 49 |
| 7 | 57 |
| 8 | 63 |
| 9 | 68 |
| 10 | 72 |
| 11 | 75 |
| 12 | 78 |
| 13 | 81 |
| 14 | 83 |
| 15 | 85 |
| 16 | 87 |
| 17 | 89 |
| 18 | 90 |
| 19 | 91 |
| 20 | 92 |
| 21 | 93 |
| 22 | 94 |
| 23 | 94 |
| 24 | 95 |

### EXAMPLE 3

120 mg Venlafaxine Delayed Controlled Release Tablets

The materials shown in Table 7 were combined to produce tablet cores for 120 mg venlafaxine delayed controlled release tablets:

TABLE 7

| Ingredients | Mg | % w/w |
|---|---|---|
| Venlafaxine Hydrochloride, USP | 135.80 | 42.4 |
| Gelling Agent¹ | 4.8 | 1.5 |
| Filler² | 169.8 | 53 |
| Lubricant³ | 9.6 | 3 |
| Purified Water⁴, USP | N/A | N/A |
| Tablet Core Weight | 320 | 100 |

¹Polyvinyl Alcohol, USP
²Lactose #315 Spray Dried, USP
³Glyceryl Behenate, NF
⁴Evaporates after drying

The tablet cores were manufactured and coated as described in Example 1 with a solution of materials shown in Table 8:

TABLE 8

| Ingredients | Mg | % w/w |
|---|---|---|
| Water-insoluble water-permeable | 27.53 | 58.58 |
| film forming polymer¹ | | |
| Water-soluble polymer² | 12.49 | 26.57 |
| Plasticizer³ | 6.98 | 14.8 |
| Solvent⁴ | N/A | N/A |
| Total Dry Solids (% weight gain) | 47 (15) | 100 |
| Tablet Cores | 320 | — |
| Total Weight of Coated Tablet | 367 | — |

¹Ethylcellulose 100, NF
²Povidone, USP
³Dibutyl Sebacate, NF
⁴Ethyl Alcohol (200 proof), USP and Isopropyl Alcohol (99%), USP, both evaporate after drying

The dissolution of the coated tablets was determined as described in Example 1 for the uncoated tablet cores. The results are presented in Table 9 as % released of the total venlafaxine in the coated tablet cores:

TABLE 9

| Time (hr) | % Released |
|---|---|
| 1 | 4 |
| 2 | 11 |
| 3 | 21 |
| 4 | 31 |
| 5 | 43 |
| 6 | 53 |
| 7 | 63 |
| 8 | 70 |
| 9 | 77 |
| 10 | 82 |
| 11 | 86 |
| 12 | 90 |
| 13 | 92 |
| 14 | 94 |
| 15 | 96 |
| 16 | 97 |
| 17 | 98 |
| 18 | 98 |
| 19 | 99 |
| 20 | 99 |
| 21 | 100 |
| 22 | 100 |
| 23 | 100 |
| 24 | 100 |

### EXAMPLE 4

180 mg Venlafaxine Delayed Controlled Release Tablets

The materials shown in Table 10 were combined to produce tablet cores for 180 mg venlafaxine delayed controlled release tablets:

TABLE 10

| Ingredients | Mg | % w/w |
|---|---|---|
| Venlafaxine Hydrochloride, USP | 203.67 | 42.4 |
| Gelling Agent¹ | 7.2 | 1.5 |
| Filler² | 254.73 | 53 |
| Lubricant³ | 14.4 | 3 |
| Purified Water⁴, USP | N/A | N/A |
| Tablet Core Weight | 480 | 100 |

¹Polyvinyl Alcohol, USP
²Lactose #315 Spray Dried, USP
³Glyceryl Behenate, NF
⁴Evaporates after drying

The tablet cores were manufactured and subsequently coated as described in Example 1 with a coating solution of materials shown in Table 11:

TABLE 11

| Ingredients | Mg | % w/w |
|---|---|---|
| Water-insoluble water-permeable | 33.966 | 58.56 |
| film forming polymer¹ | | |
| Water-soluble polymer² | 15.39 | 26.53 |
| Plasticizer³ | 8.64 | 14.8 |
| Solvent⁴ | N/A | N/A |
| Total Dry Solids (% weight gain) | 58 (12) | 100 |
| Tablet Cores | 480 | — |
| Total Weight of Coated Tablet | 538 | — |

¹Ethylcellulose 100, NF
²Povidone, USP
³Dibutyl Sebacate, NF
⁴Ethyl Alcohol (200 proof), USP and Isopropyl Alcohol (99%), USP, both evaporate after drying

The dissolution of the coated tablets was determined as described in Example 1 for the uncoated tablet cores. The results are presented in Table 12 as % released of the total venlafaxine hydrochloride in the coated tablet cores:

TABLE 12

| Time (hr) | % Released |
|---|---|
| 1 | 3 |
| 2 | 11 |
| 3 | 20 |
| 4 | 30 |
| 5 | 40 |
| 6 | 50 |
| 7 | 60 |
| 8 | 68 |
| 9 | 74 |
| 10 | 80 |
| 11 | 84 |
| 12 | 87 |
| 13 | 90 |
| 14 | 92 |
| 15 | 94 |
| 16 | 95 |
| 17 | 96 |
| 18 | 97 |
| 19 | 97 |
| 20 | 98 |
| 21 | 98 |
| 22 | 98 |
| 23 | 98 |
| 24 | 98 |

1. A delayed controlled release pharmaceutical composition for oral administration suitable for once daily dosing comprising:
a) a core comprising by weight of the core dry weight from about 10% to about 90% of at least one form of venlafaxine selected from the group consisting of venlafaxine, a pharmaceutically acceptable salt of venlafaxine, an active metabolite of venlafaxine, a pharmaceutically acceptable salt of an active metabolite of venlafaxine, and combinations thereof, less than 10% of a gelling agent, and optional conventional excipients;
b) a modified release coat substantially surrounding said core, said coat comprising by weight of the coat dry weight about 60% ethylcellulose, about 25% polyvinypyrrolidone and about 15% of a plasticizer;
wherein said composition provides a delayed controlled release of said at least one form of venlafaxine such that no more that 20% of the at least one form of venlafaxine is released after about 2 hours, about 15% to about 45% of the at least one form of venlafaxine is released after about 4 hours, about 55% to about 85% of the at least one form of venlafaxine is released after about 8 hours, no less than about 65% of the at least one form of venlafaxine is released after about 12 hours and no less than about 80% of the at least one form of venlafaxine is released after about 16 hours when tested using USP Apparatus 1 in 1000 ml of pH 6.8 phosphate buffer at 75 rpm at 37° C.±0.5° C.

2. The delayed release composition of claim 1 which optionally contains at least one other coating.

3. The delayed release composition of claim 1 wherein the plasticizer comprises a polyol, organic, ester, oil or glyceride.

4. The delayed release composition of claim 1 wherein the pharmaceutically acceptable salt of venlafaxine comprises acetic, benzenesulfonic benzoic, camphorsulfonic, citric, ethensulfonic, fumaric, gluconic, glutamic, hydrobomic, hydrochloric isethionic, lactic, maleic, malic, mandelic, methansulfonic, mucic, nitric, pamoic, pantothenic, phosphoric, succinic, sulfuric, tartaric acid, p-toluenesunfonic and the like.

5. The delayed release composition of claim 4 wherein the salt is the hydrochloric salt.

6. The delayed release composition of claim 1 which comprises from 20 to 200 mg of venlafaxine.

7. The delayed release composition of claim 6 which comprises 30 mg of venlafaxine.

8. A method of treating depression in a patient in need thereof comprising administering once daily a delayed release composition according to claim 1.

9. The modified release composition of the at least one form of venlafaxine is a delayed controlled release pharmaceutical composition for oral administration suitable for once daily dosing comprising: a) a core comprising weight of the core dry weight from about 10% to about 90% of at least one form of venlafaxine selected from the group consisting of venlafaxine, a pharmaceutically acceptable salt of an active metabolite of venlafaxine, and combinations thereof, less than 10% of a gelling agent, and optional conventional excipients; and b) a modified release coat substantially surround said core, said coating comprising by weight of the coat dry weight from about 20% to about 85% of a water-insoluble water-permeable film-forming polymer, from about 10% to about 75% of a water-soluble polymer or substance and from about 3% to about 40% of a plasticizer; wherein said composition provides a delayed controlled release of said at least one form of venlafaxine such that no more that 20% of the at least one form of venlafaxine is released after about 2 hours, about 15% to about 45% of the at least one form of venlafaxine is released after about 4 hours, about 55% to about 85% of the at least one form of venlafaxine is released after about 12 hours and no less than about 80% of the at least one form of Venlafaxine is released after about 16 hours when tested using USP Apparatus 1 in 1000 ml of pH 6.8 phosphate buffer at 75 rpm at 37° C.±0.5° C.

10. A method of treating depression in a patient in need thereof comprising administering once daily a delayed release composition according to claim 9.
